(19) United States
(12) Patent Application Publication
(10) Pub. No.: US 2006/0008904 A1
(43) Pub. Date: Jan. 12, 2006

(54) Cell culture method and apparatus thereof

(75) Inventors: Junzo Tanaka, Tsukuba-shi (JP); Toshiyuki Ikoma, Tsukuba-shi (JP); Takeshi Muneta, Hino-shi (JP)

Correspondence Address: WENDEROTH, LIND & PONACK, L.L.P., 2033 K STREET N. W., SUITE 800, WASHINGTON, DC, 20006-1021, US

(73) Assignee: NATIONAL INSTITUTE FOR MATERIALS SCIENCE

(21) Appl. No.: 10/525,877

(22) PCT Filed: Aug. 26, 2003
(86) PCT No.: PCT/JP03/10766

§ 371 Date: Jul. 27, 2005

(30) Foreign Application Priority Data
Aug. 26, 2002 (JP) 2002-245987

(51) Int. Cl. C12N 5/00; C12M 3/00

(52) U.S. Cl. 435/394; 435/286.7; 435/289.1; 435/298.2

(57) ABSTRACT

By rotating a cell-culture container while controlling the temperature and the gaseous atmosphere, cells are cultured under the application of hydrostatic pressure due to centrifugal force, and according to this cell culture, cells are efficiently activated and cultured while inhibiting dedifferentiation.

### TECHNICAL FIELD

The invention of the present application relates a method for cell culture and an apparatus thereof. More specifically, it relates to a novel method for cell culture capable of simply and efficiently obtaining cells specifically useful for regenerative medicine, as well as an apparatus thereof.

### BACKGROUND ART

In cell culture, since during the process to lose the function in a mono layer cell culture growing cells only on the surface layer of a culture medium cells of a certain type become dedifferentiated, to prevent this a method of adding growth factors, chemicals, etc. to control dedifferentiation or a method of activating the cell function by mechanical stimulation in conventionally utililzed.

Among the methods of controlling cell dedifferentiation by mechanical stimulation are a method using hydrostatic pressure (Effects of physical stimulation on chondrogenesis in vitro, Materials Science and Engineering C6(1998)301-306) and a high density culture technique utilizing centrifugal force.

However, existent methods of utilizing hydrostatic pressure have the drawback that they are not only expensive but also the culture apparatus is large and requires much space. On the other hand, in the existent method utilizing the centrifugal force, since the centrifugal force is applied at a normal temperature after previous culture in an incubator, it involves a problem that it is impossible to have a culture environment continuously applying stimulations for a long time, or periodically changing the stimulation. In addition, in the existent method of utilizing the centrifugal force, control of the temperature and the surrounding atmosphere was impossible.

In view of the above, it is a goal of the invention of the present application to solve the problems described above and to provide a new method capable of simple and efficient cell culture while suppressing dedifferentiation mechanically by stimulations, as well as an apparatus thereof.

### DISCLOSURE OF THE INVENTION

The invention of the application, in order to solve the foregoing problems, provides firstly a method for cell culture which continuously controls dynamic conditions by application of hydrostatic pressure on a culture liquid in a condition for culturing cells by centrifugal force, thereby giving stimulation to the cells. Secondarily, it provides a method for cell culture wherein for the controlling of dynamic culture condition by application of hydrostatic pressure, application of the hydrostatic pressure to the cells is periodically changed or maintained for a certain period of time by application of centrifugal force. Thirdly, it provides a method for cell culture as described above wherein the hydrostatic pressure is applied in a range of 60 MPa or less; fourthly, it provides a method for cell culture described above wherein the hydrostatic pressure is applied within a range from 0.5 sec to 6 weeks, fifthly, it provides a method for cell culture described above wherein application of the hydrostatic pressure is conducted through control of the number of rotation of a centrifugator and, sixthly, it provides a method for cell culture described above wherein the temperature and the atmosphere are controlled. Then, seventhly, the invention of the present application provides a method for cell culture as described above wherein the cells are cultured together with various kinds of biomaterials.

Further, eighthly, the invention of the present application provides an apparatus for the cell culture having a cell-culturing device supported by a rotational shaft in a sealed container for providing cells with a hydrostatic pressure by centrifugal rotation, ninthly, it provides an apparatus for the cell culture as described above, wherein comprising a control mechanism for controlling the rotation time and the rotation speed of the cell culturing device, tenthly, it provides an apparatus for the cell culture as described above wherein the number of rotations is controllable within a range from 10 to 25000 rpm for providing a hydrostatic pressure at 60 MPa or less and, eleventhly, it provides an apparatus for the cell culture as described above wherein the inside of the cell culturing device is divided such that a plurality types of cell cultures can be conducted simultaneously.

The invention of the application provides, twelfthly, an apparatus for the cell culture as described above wherein an injection port and an exhaust port for an atmospheric gas into and out of the sealed container, and a control mechanism for injecting and exhausting the atmospheric gas are provided, and, thirteenthly, it provides an apparatus for the cell culture as described above wherein a control mechanism for the temperature in the sealed container is provided.

In the growth of organisms, stem cells, which are undifferentiated and not specified as to function, are successively specialized with regard to function while repeating differentiation into various organs or tissues. However, the cell differentiation in the organism is not always conducted under identical conditions, circumstances such as the pressure or the temperature in the organism undergoing change.

The invention of the application takes this point into account and is intended to culture cells under conditions approximating actual in-vivo conditions. The gist of the invention of the application intending to create an environment similar to the organism has a feature of culturing the cells while controlling culture environment by the centrifugal application of a hydrostatic pressure as described above.

Such a feature is based on the knowledge that the liquids inside a specimen tube rotationally driven by a centrifugal force always receive a centrifugal force and a hydrostatic pressure, the level of the centrifugal force and the hydrostatic pressure can be controlled simply by controlling the number of rotations of a centrifugator, and that it is possible to give stimulations to the cells and suppress cell dedifferentiation by executing this control during culture of the cells.

Further, the invention of the application also achieves control of the temperature and the atmosphere in the culture. The invention according to the application, enables growth of cells while controlling activation and dedifferentiation as described above and makes it possible to obtain in a short period of time various kinds of organism tissues including the extra-cellular matrix extending between cells, which maintains health of cells and in which is written information to suppress growth and dedifferentiation. This is promising for application in regenerative medicine.

For example, by periodically applying hydrostatic pressure of from 0.1 MPa to 30 MPa to the cells by rotating the cell culture apparatus of the invention, not only the in-vivo circumstance equal to the rhythm of human walking but also can control high gravitational force (for example, as in the environment in the deep sea). Then, by periodically changing hydrostatic pressure and the centrifugal force on the cells, thereby enhancing the cell activity, cell agglomerates are formed, and the hydrostatic pressure controls the cell metabolism to control the dedifferentiation.

The features as described above cannot be attained by or even induced from the existent methods of utilizing the hydrostatic pressure or the existent methods of exerting the centrifugal force after previously conducting the culture.

### BRIEF DESCRIPTION OF THE DRAWINGS

FIG. 1 shows a cross sectional constitutional view exemplifying the outline of an apparatus for the cell culture according to the invention of the present application.

FIG. 2 shows a schematic view exemplifying a test container in a cell culturing device and an exerted force.

FIG. 3 shows a correlation graph between hydrostatic pressure and time exemplifying the state of keeping an identical hydrostatic pressure.

FIG. 4 shows a correlation graph between hydrostatic pressure and time exemplifying the state of periodically changing the hydrostatic pressure.

FIG. 5 shows a graph showing the increase for the number of cells in the invention utilizing centrifugal force of the present application and in a conventional high density culturing method and mono-layer culturing method.

FIG. 6 shows a photograph by a phase-contrast microscope of cells cultured for one week by a method of applying a hydrostatic pressure of the invention according to the present application.

FIG. 7 shows a photograph by a phase contrast microscope for cells cultured for 1 week by a conventional mono-layer culturing method.

References in the drawings shown the followings.

1 . . . cell-culturing device

2 . . . lid for the cell-culturing device

3 . . . rotational shaft

4 . . . motor

5 . . . heater

6 . . . sealed container

7 . . . culturing tube

8 . . . distance between a rotary shaft and a culture solution surface

9 . . . centrifugal force

C . . . control mechanism

G . . . carbon dioxide-incorporated steam

### BEST MODE FOR CARRYING OUT THE INVENTION

The invention of the present application has features as described above and embodiments thereof are to be described below.

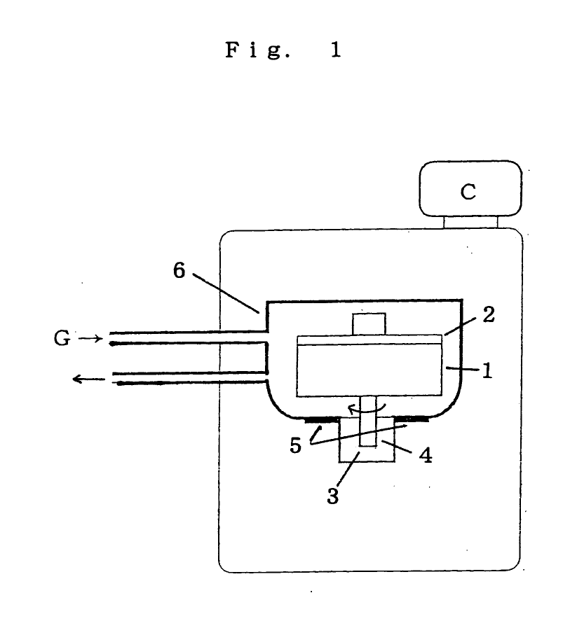

At first, referring to a method for cell culture and the outline of an apparatus according to the invent ion of the present application with reference to the drawings, FIG. 1 shows an overall view of a cell culturing apparatus in which cells and a culturing liquid can be freely filled and taken out by opening and closure of a lid (2). The cell-culturing device (1) is supported in a sealed container (6) having a heating mechanism (5), by a rotational shaft (4) connected with a motor (3). Further, steam (G) incorporated with gaseous carbon dioxide is circulated to the sealed container (6). The atmospheric gas is injected by way of a channel formed in the lid (2) into the cell-culturing device (1) and exhausted therefrom by the opening and closure of the lid (2). Then, the rotational speed, the rotational time, and the temperature and the atmospheric gas for the cell-culturing device (1) are controlled by a previously set control mechanism (C).

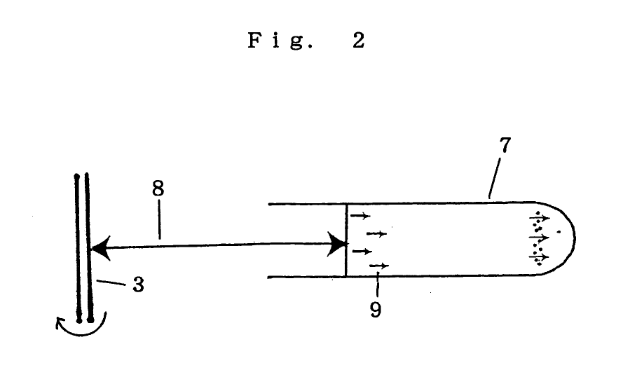

FIG. 2 is a schematic view partially exemplifying the inside of the cell-culturing device (1) of FIG. 1, in which a hydrostatic pressure can be applied by centrifugal force (9) to the cells in the container (7) of a test tube-like configuration connected with the rotational shaft (3), created by rotating the same after charging cells and culturing liquid.

The rotational speed and the rotational time in this case are controlled by the control mechanism (C) in FIG. 1.

Thus, the level of the hydrostatic pressure, the centrifugal force exerted on the cells, and the time thereof can be freely controlled.

The level of the hydrostatic pressure and the centrifugal force are represented, for example, as shown below.

TABLE 1

| | |
|---|---|
| Hydrostatic pressure | Centrifugal Force |
| depends on the height of solution | (exerted on cell · material) |
| HP (atm cm²/Kg) = 5.48 × | RCF(G) = 11.18 × r(cm) × |
| 10⁻⁹ × D × Q² (r² − r ₘₑₙ²) | (Q/1000)² |
| D: Average density of | r: distance between granules |
| solution (g ml⁻¹ | and center |
| r: distance from the | Q: rotational speed (rpm) |
| center (cm) | |

rmem: distance from the liquid surface to the center of rotation (cm)

The relation between the number of rotations and the hydrostatic pressure is exemplified as below.

TABLE 2

| | | | | | | |
|---|---|---|---|---|---|---|
| Cell culture liquid | 1.14 | g/cm⁻¹ | | | | |
| (MEM) | | | | | | |
| Height of solution | 2 | cm | | | | |
| Number of rotation | 3000 | rpm | 5000 | rpm | 10,000 | rpm |
| Hydrostatic pressure | 2 | Mpa | 5.6 | Mpa | 22.5 | Mpa |

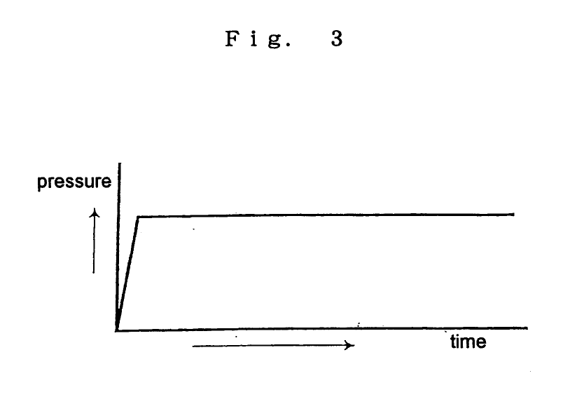

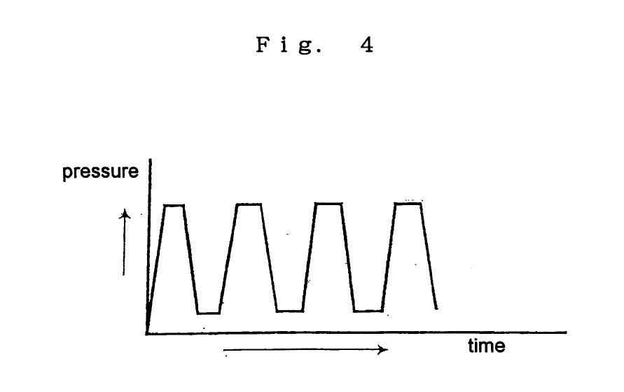

FIG. 3 shows the hydrostatic pressure in the container (7) when the rotational speed is maintained for a certain period and FIG. 4 shows the change of the hydrostatic pressure in the container (7) when the rotational speed is changed intermittently. The invention of the present application is conducted due to application patterns of the hydrostatic pressure in the container which are shown in a simplified manner in FIG. 3 and FIG. 4 or combination of such application patterns, and the setting of the conditions for this combination is based on the following facts.

The specific number of rotations for forming the hydrostatic pressure in the case of culturing similar to in-vivo conditions is, preferably, from 10 to 25,000 rpm and particular, preferably from 10 to 23,000 rpm because when the number of rotation exceeds 25,000 rpm, the hydrostatic pressure exceeds 60 Mpa and possibly destroys the cells.

Further, the application time of the hydrostatic pressure is limited to a range of from 0.5 sec to 6 weeks. This is because the cycle of pressure exerted on a human body during vigorous motion is considered to be 0.5 sec and on the other hand, the culturing period of cells used upon transplantation to a human body is about within four weeks.

Moreover, the temperature is preferably from about 0° C. to 50° C. which is within the range of living organisms, and a temperature about from 25° C. to 40° C. which is the usual body temperature of organisms is particularly preferred.

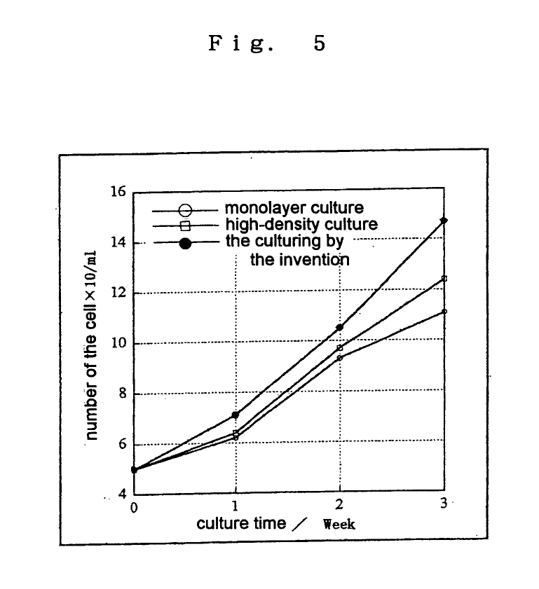

FIG. 5 shows comparison between the cell culture under the conditions described above and cell culture according to a conventional method. FIG. 5 shows those cultured according to the invention of the present application (shown by solid circles), those grown by conventional high density culture (indicated by blank squares), and those of mono-layer culture (indicated by blank circles), cultured for 1 week to 3 weeks, the number of cells being plotted. As is apparent from FIG. 5, the cell growth ratio in the invention of the present application is 15% greater than the existent high density culture and 30% greater than the existent mono layer culture after three weeks.

Examples of the invention according to the application and the monolayer culture are shown below and described more specifically.

Off course, the invention is not restricted by the following examples.

### EXAMPLE

Cartilage cells (5×10⁴ cells/ml) were grown by centrifugal culture in a culture solution of MEM (Minimum Essential Medium) at a temperature of 25° C. and in atmospheric air for 1 week. The centrifugal conditions were: rotating for 30 min twice a day at a 1000 rpm, conducted three times per week. On the other hand, a monolayer culture was conducted as a control.

As a result of centrifugal culture, the final number of cells was 10.2×10⁴ cells/ml, whereas it was 8.4×10⁴ cells/ml in the monolayer culture; thus the centrifugal culture improves the growth ratio by at least 20%.

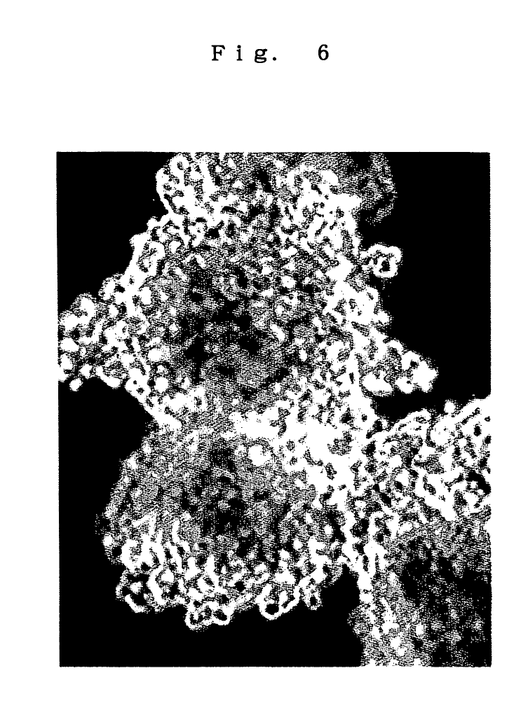

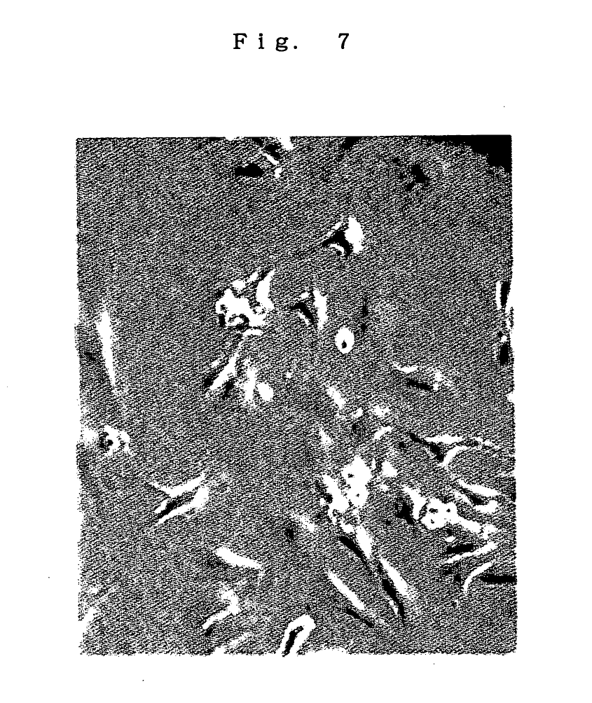

Further, FIG. 6 and FIG. 7 show the states in comparison by phase contrast microscopic photograph.

It is clearly shown that the number of cells grown by the method for centrifugal culture according to the invention of the present application (FIG. 6) is greater than the number of cells grown by the mono layer culture method (FIG. 7). Further, spherical shapes inherent to the cartilage cells remain in the centrifugal culture, whereas they were dedifferentiated into cells of advanced fibrillation in the monolayer culture.

From the foregoing result, it was confirmed that the centrifugal culture can increase the growth ratio of cells and also suppress dedifferentiation of cells.

### INDUSTRIAL APPLICABILITY

As has been described above in detail, the invention of the present application not only can efficiently manufacture substrate materials for the cell growth in regenerative medicine by activating the cells and suppressing dedifferentiation but also can grow, conveniently and efficiently, specific cells for the individual patients in so-called tailor-made treatment.

1-13. (canceled)

14. A method for cell culture which continuously controls dynamic conditions by application of hydrostatic pressure on a culture liquid in an atmospheric air condition or a gaseous atmosphere condition for culturing cells by centrifugal force, thereby giving stimulation to the cells.

15. A method for cell culture according to claim 14, wherein for the controlling of dynamic culture conditions by application of hydrostatic pressure, application of the hydrostatic pressure to the cells is periodically changed or maintained for a certain period of time by application of centrifugal force.

16. A method for cell culture according to claim 14, wherein the hydrostatic pressure is applied in a range of 60 MPa or less.

17. A method for cell culture according to claim 14, wherein the hydrostatic pressure is applied within a range from 0.5 sec to 6 weeks.

18. A method for cell culture according to claim 14, wherein the application of the hydrostatic pressure is conducted by controlling the number of rotations of a centrifugator.

19. A method for cell culture according to claim 14, wherein the temperature and the atmosphere are controlled.

20. A method for cell culture according to claim 14, wherein cells are cultured together with various kinds of biomaterials.

21. An apparatus for cell culture having a cell-culturing device supported by a rotational shaft in a sealed container for providing cells with hydrostatic pressure by centrifugal rotation.

22. An apparatus for cell culture according to claim 21, wherein comprising a control mechanism for controlling the rotation time and the rotation speed of the cell-culturing device.

23. An apparatus for cell culture according to claim 21, wherein the number of rotations is controllable within a range from 10 to 25,000 rpm for providing the hydrostatic pressure of 60 MPa or less.

24. An apparatus for cell culture according to claim 21, wherein the inside of the cell culturing device is divided so that a plurality of types of cells can be cultured simultaneously.

25. An apparatus for cell culture according to claim 21, wherein an injection port and an exhaust port for the gaseous atmosphere into and out of the sealed container, and a control mechanism for injecting and exhausting the atmospheric gas are provided.

26. An apparatus for cell culture according to claim 21, wherein a control mechanism for the temperature in the sealed container is provided.

27. A method for cell culture according to claim 15, wherein the hydrostatic pressure is applied in a range of 60 MPa or less.

28. A method for cell culture according to claim 15, wherein the hydrostatic pressure is applied within a range from 0.5 sec to 6 weeks.

29. A method for cell culture according to claim 16, wherein the hydrostatic pressure is applied within a range from 0.5 sec to 6 weeks.

30. A method for cell culture according to claim 27, wherein the hydrostatic pressure is applied within a range from 0.5 sec to 6 weeks.

31. A method for cell culture according to claim 15, wherein the application of the hydrostatic pressure is conducted by controlling the number of rotations of a centrifugator.

32. A method for cell culture according to claim 16, wherein the application of the hydrostatic pressure is conducted by controlling the number of rotations of a centrifugator.

33. A method for cell culture according to claim 27, wherein the application of the hydrostatic pressure is conducted by controlling the number of rotations of a centrifugator.
